(19) United States
(12) Plant Patent Application Publication
(10) Pub. No.: US 2006/0048257 P1
(43) Pub. Date: Mar. 2, 2006

(54) Variety of cordyline plant named "Bra01'

(76) Inventor: Peter Brauns, Queensland (AU)

Correspondence Address: FULWIDER PATTON LEE & UTECHT, LLP, 200 OCEANGATE, SUITE 1550, LONG BEACH, CA, 90802, US

(21) Appl. No.: 10/929,291

(22) Filed: Aug. 30, 2004

(51) Int. Cl. A01H 5/00

(52) U.S. Cl. PLT/373

(57) ABSTRACT

A new and distinct cultivar of Cordyline plant named ‘BRA01’ characterized by a small clump forming growth habit with deep purple-black foliage and a relatively short intemodel space.

### BACKGROUND OF THE NEW PLANT

The present invention relates to a new and distinct cultivar of Cordyline plant, botanically known as Cordyline fruiticosa and further known by the varietal name ‘BRA01’. This new variety is also sold under the trade name CARUBA BLACK. The new variety was discovered as a sport of a wild green Cordyline fruiticosa and collected by Michael Yappi in the Western Highlands region of New Guinea in Mt. Hagen. The sport was then provided to a plant collector, Michael Ferrero of Cairns, Queensland Australia, and imported to plant breeder Peter Brauns of Plant Source Australia who performed further trials and selections using vegetative reproduction techniques. The parental cultivar is a green leaved Cordyline fruiticosa (unpatented). The cuttings were planted out and plants with desirable ornamental characteristics were selected. The cultivar was isolated by the inventor in Australia by selecting primarily on the basis of compact growth habit, foliage color, and suitability as a garden or container grown plant.

Following selection of the plant, the cultivar was propagated vegetatively through cutting and division at Edmonton, Queensland, Australia. Continued observation of the vegetative divisions resulting from asexual reproduction taken at Edmonton, Queensland, Australia have confirmed that the distinguishing features of this new cultivar came true, remain stable and are retained through successive propagation.

The closest comparator to ‘BRA01’ known to applicant is Cordyline fruiticosa ‘Hawaiian Flag’ (unpatented) which is a clump forming plant with green foliage and some purple and yellow variegations, a mean width of 92 mm, and a mean length of 389 mm excluding the petiole. The parent wild green C. fruiticosa has green foliage and a plant size up to 1.8 meters.

### SUMMARY OF THE INVENTION

Plants of the cultivar ‘BRA01’ have not been observed under all possible environmental and cultural conditions. The phenotype may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘BRA01’. These characteristics distinguish ‘BRA01’ as a new and distinct cultivar from its closest comparator Cordyline fruiticosa ‘Hawaiian Flag’:

1. Purple-black leaves with deep green hues;

2. Compact growth habit up to 1.2 meters;

3. Shorter leaf length;

4. Smaller leaf width; and

5. Shorter intemodal space.

Compared to other plants of the Cordyline genus, the new Cordyline has a distinctive foliage with purple-black leaves having deep green hues.

### BRIEF DESCRIPTION OF THE DRAWINGS

The invention is depicted in the accompanying colored photographs in which:

FIG. 1 shows a potted plant illustrating the characteristic foliage.

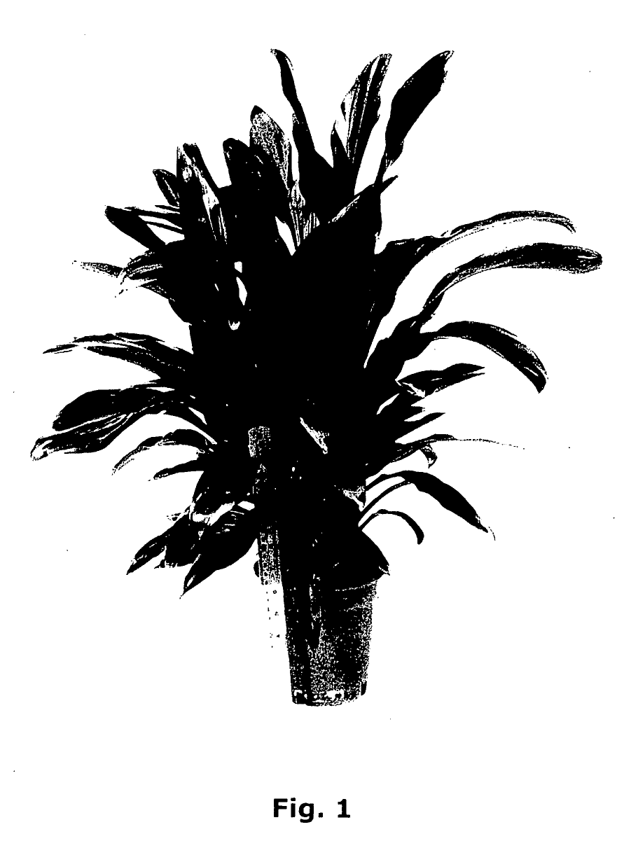

The photograph depicts the overall appearance of the new Cordyline, showing the colors as true as it is reasonably possible to obtain in colored reproductions of this type. Colors in the photograph may differ slightly from the color values cited in the Detailed Botanical Description which more accurately describe the actual colors of the new Cordyline.

### DETAILED BOTANICAL DESCRIPTION OF THE NEW PLANT

The following detailed description sets forth characteristics of the new cultivar. The data which defines each characteristic was collected from asexual reproductions of the plant grown in an open greenhouse with irrigation provided by drip feeding in a hydroponic system, and carried out by the observer at Edmonton, Queensland, Australia. The plant histories of Cordyline ‘BRA01’ were taken on 2 pots with 3 plants each and taking place in approximately January 2004 through June 2004.

In the following table of plant characteristics, color references are made to The Royal Horticultural Society of London Colour Chart, 1995 Edition. The measurements were taken from mature leaves.

### The Plant

Classification:
   Botanical.—Cordyline fruiticosa.
   Varietal name.—‘BRA01’.
   Parentage.—Sport of wild green Cordyline fruiticosa.
   Asexual propagation.—root division and cuttings.
Form:
   Plant growth rate.—Moderate.
   Growth habit.—Upright, clumping, and compact.
   Plant density.—Bushy.
   Growth type.—Clumping.
   Mean plant height.—650 mm.
   Mean plant width.—550 mm.
   Stem.—Scapes — branching arrangement Mean stem diameter — 9.75 mm.
Leaves:
   Leaf color.—Purple-black group RHS 200A with deep green hues.
   Leaf shape.—Petiolate with lobed leaves.
   Mean Leaf width (mm).—69.6 mm.
   Mean Leaf length (mm).—286 mm (excluding petiole).
   Leaf characteristics.—Apex — V-shaped Margin — Slight undulations Texture — Smooth, semi-glossy Shape — Lobed with a long petiole.
   Petiole mean length.—105.86 mm.
   Petiole mean width.—18.62 mm.
   Petiole shape.—Curves upward, not joining.
   Petiole color (inside).—144B.
   Petiole color (outside).—200B with a Burgundy hue.
Inflorescence: May not flower in temperate zones.

### General Characteristics

‘BRA01’ is primarily distinguished from other cultivars of Cordyline genus by its purple-black foliage, compact growth habit, and shorter internodal space. ‘BRA01’ is a bushy clump forming plant with an upright compact growth habit wherein the leaves are petiolate and lobed with a smooth, semi-glossy texture and slight undulations on their margins.

What is claimed is:

1. A new and distinct Cordyline fruticosa plant named ‘BRA01’ substantially as illustrated and described.
